(19) United States
(12) Patent Application Publication
(10) Pub. No.: US 2018/0035984 A1
(43) Pub. Date: Feb. 8, 2018

(54) BIOPSY UNIT

(71) Applicant: SIEMENS HEALTHCARE GMBH, ERLANGEN (DE)

(72) Inventors: KARSTEN SCHMITT, ERLANGEN (DE); STEFAN VEITENHANSL, NUERNBERG (DE)

(21) Appl. No.: 15/667,819

(22) Filed: Aug. 3, 2017

(30) Foreign Application Priority Data
Aug. 3, 2016 (DE) 10 2016 214 319.0

(51) Int. Cl. A61B 10/02; A61B 6/00; A61B 90/00

(52) U.S. Cl. CPC A61B 10/0233; A61B 90/39; A61B 6/4007; A61B 6/022

(57) ABSTRACT

A biopsy unit is configured with a marker unit having at least one marker such that a treatment point can be displayed in accordance with the guidance of a biopsy needle on the surface of an object to be examined. The coordinates of a determined tissue alteration within an object to be examined are used in order to align the marker unit.

### CROSS-REFERENCE TO RELATED APPLICATION

This application claims the priority, under 35 U.S.C. §119, of German application DE 10 2016 214 319.0, filed Aug. 3, 2016; the prior application is herewith incorporated by reference in its entirety.

### BACKGROUND OF THE INVENTION

### Field of the Invention

In order to perform a biopsy, two or for a tomosynthesis imaging method a plurality of X-rays are created for stereo imaging purposes for example in order to capture possible tissue alterations in a breast of a patient. Tissue alterations are localized in the 2D or 3D X-ray image data with the aid of image processing algorithms and the coordinates of the selected tissue alterations are determined. In order to enable the physician to carry out further treatment steps for removal of a tissue alteration within the breast of the patient, a biopsy needle of a biopsy needle unit fixed in a biopsy needle holder is aligned with the tissue alteration. In this manner the treating physician can gain an understanding of where analgesia and an incision for taking a biopsy from the breast of the patient need to be targeted. Prior to local anesthesia and making a cut into the skin it is necessary to determine and disinfect the puncture site for the biopsy needle. In order to perform the preparatory treatment steps for taking the biopsy, following a first alignment with a tissue alteration to be examined on a prioritized basis the biopsy needle holder together with the biopsy needle unit is returned to a type of parked position and the biopsy needle unit is removed from the biopsy needle holder again in order to obtain additional space for the treatment steps to be undertaken by the physician. In addition to the disadvantage that hitherto it has not been possible to effect an exact marking or has been possible only to effect an approximate marking on account of the lack of treatment space, this is accompanied by the further disadvantage involving a further time requirement that the biopsy needle unit for taking the biopsy must be positioned a second time to the surface of the breast of the patient and the biopsy needle must again be aligned with the tissue alteration to be examined.

A method and an orientation device for a medical instrument are known from the published, non-prosecuted German patent application DE 10 2010 031 943 A1 in such a way that a light source for displaying a treatment point on an object and an inclinometer are to be provided on the medical instrument for the purpose of guiding the medical instrument within the object in accordance with the medical intervention.

### SUMMARY OF THE INVENTION

It is accordingly an object of the invention to provide biopsy unit that overcomes the above-mentioned disadvantages of the prior art devices of this general type.

The biopsy unit is configured with an alignable biopsy needle holder which accommodates a lockable biopsy needle unit for taking a biopsy in such a manner and having a marker unit containing at least one marking device for specifying a treatment point to be displayed on the surface of an object. The coordinates of a determined tissue alteration within the object to be examined are used in order to align the marker unit and in order to align the marking device the biopsy needle holder for the biopsy needle unit is configured such that at least one marking device is alignable such that the one or more marking devices is/are aligned with the tissue alteration within an object and marks/mark the treatment point on the surface of the object.

The invention offers the advantage that an exact marking for preparatory treatment steps for taking a biopsy can be performed by using a marker unit associated with the biopsy unit.

The invention offers the advantage that following a local determination of a tissue alteration the preparatory treatment steps for taking a biopsy can be performed immediately.

The invention offers the advantage that the first alignment of the biopsy needle mentioned in the introduction is dispensed with and the biopsy needle unit with the biopsy needle inserted is positioned and aligned exactly only after the treatment steps performed by the physician for taking a biopsy.

The invention offers the advantage that a laser beam emitted from the marker unit is aligned in accordance with the alignment of the longitudinal axis of the aligned biopsy needle and the laser beam exactly predefines the alignment of the biopsy needle for taking a biopsy.

The invention offers the advantage that a maximum treatment space for the treatment steps to be undertaken is made available to the physician in particular by means of a marker unit having optical marking measures.

The invention offers the advantage that the biopsy needle unit only needs to be positioned to the patient once in order to take the biopsy.

The invention offers the advantage that the entire procedure of taking a biopsy is shortened.

Other features which are considered as characteristic for the invention are set forth in the appended claims.

Although the invention is illustrated and described herein as embodied in a biopsy unit, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is an illustration showing a first embodiment of a biopsy unit according to the invention; and

FIG. 2 is an illustration showing a second embodiment of the biopsy unit.

### DETAILED DESCRIPTION OF THE INVENTION

A biopsy unit is configured with a marker unit containing least one marking device such that at least one treatment point is displayed in accordance with the guidance and alignment of a biopsy needle on the surface of an object to be examined.

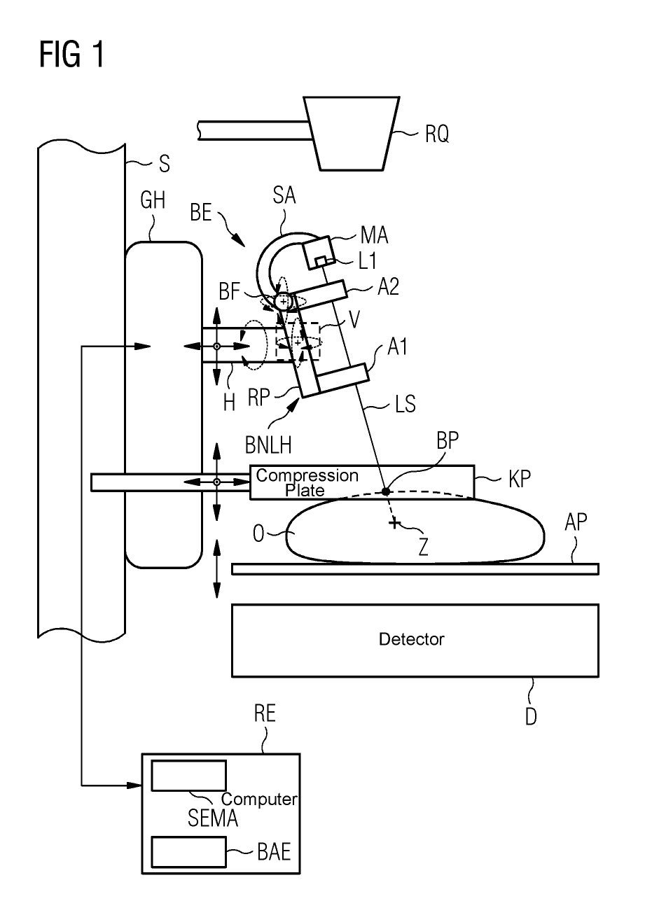

Referring now to the figures of the drawings in detail and first, particularly to FIG. 1 thereof, there is shown a part of a mammography system having an X-ray source RQ which can be fixed on a stand S and can be moved along a trajectory and having a detector unit D which is adjustable at least in height can be seen. Furthermore, a biopsy unit BE is illustrated schematically from the side. The biopsy unit BE is likewise depicted schematically, having a holding arm H designed for the positioning of a biopsy needle holder BNLH and having a housing GH accommodating the control elements for the positionable holding arm H. The units are coordinated or controlled in each case by a computer unit RE associated therewith. For controlling the biopsy unit BE, at least parts of an image analysis unit BAE and also a control unit SEMA for a marker unit MA are provided. The image analysis unit BAE can also be a component of further diagnostic units which are in communication are with the mammography system. By means of the image analysis unit BAE and the X-ray images required for the purpose the tissue of the object O fixed here on a working platform AP by means of a compression unit KP, for example the breast of a patient, is searched for abnormalities and should any such abnormalities be identified the coordinates of the tissue alteration relating to the analyzed image material are saved and where applicable marked in the 2D or 3D image material. The coordinates determined are transferred to the control unit SEMA for the alignment of the marker unit MA. A holding arm H which can be moved at least in the vertical and/or horizontal direction by way of control elements positions the biopsy needle holder BNLH. The housing GH of the biopsy unit BE can be positioned and fixed on the stand S. A coupling of the biopsy unit BE with the movement of the compression plate KP is possible. A connection element V which can be aligned at least in one axis and is also lockable can optionally be provided between holding arm H and biopsy needle holder BNLH. The biopsy needle holder BNLH has a first and a second arm A1, A2 arranged on a rear wall plate RP. The first and the second arms A1, A2 serve to at least partially surround the biopsy needle unit and enable a solid connection to the biopsy needle holder BNLH which can be positioned manually or electronically. The biopsy needle unit can also be moved and positioned as desired and fixed by a carriage on semi-circular guide elements. The biopsy needle holder BNLH shown in FIG. 1 can be moved, positioned and aligned in any desired direction by adjusting elements which can be driven by motor. The marker unit MA is connected by way of a swivel arm SA to the biopsy needle holder BNLH. The swivel arm SA is connected at its first end to the marker unit MA and at its second end to the biopsy needle holder BNLH. The swivel arm SA is configured in such a way that the marking device of the marker unit MA arranged at the first end thereof displays a treatment point BP on the breast O of a patient. An optical marking device and also a marking device delivering a marking substance can be used as the marking device. The marking device delivering the marking substance can be a stylus capable of extending telescopically from the marker unit MA. In this present embodiment the marking device is a laser L1 emitting a laser beam LS. The laser L1, also referred to as first laser, is designed as a point-type laser. In addition to the treatment point BP, the laser beam LS from the first laser L1 arranged in the marker unit MA also gives the treating physician the alignment of the biopsy needle needed for taking the biopsy. The laser beam LS from the first laser L1 lies on the longitudinal axis of the biopsy needle of a biopsy needle unit to be inserted at a later point in time into the biopsy needle holder BNLH and marks the treatment point BP on the skin of the patient. In order to align the laser beam LS from the first laser L1, which is designed as a point-type laser, with the treatment point BP, depending on the calculated localization of the tissue alteration Z the positioning motors for the holding arm H and thereby the biopsy needle fixed in the biopsy needle holder BNLH can be moved or aligned in accordance with the indicated three axes of the holding arm H. In a further embodiment for fine adjustment the point-type laser L1 can additionally be aligned or adjusted mechanically in the marker unit MA such that its alignment corresponds exactly to the subsequent needle guidance of the biopsy needle. An adjustment of the marker unit MA or of the point-type laser can be effected through the control unit SEMA likewise arranged in the computer unit RE. These control signals initiated by the control unit SEMA control the positioning motors for the holding arm H which are arranged in the housing GH. They serve for example to adjust the height of the holding arm H and the distance from the patient and to align the biopsy needle holder BNLH in such a manner until an optimum path is found, using the determined target coordinates Z, for taking the biopsy. The laser light LS emitted from the laser L1 gives the alignment of the biopsy needle. The holding arm H and the biopsy needle holder BNLH can then be moved or manually aligned in accordance with the coordinates of the identified tissue alteration in such a manner that the biopsy needle for example takes the shortest path through the tissue of the fixed object O to the localized tissue alteration Z. In order to align the point-type laser L1 in the marker unit MA the biopsy needle holder BNLH is aligned with a tissue alteration as described above. In the case of an electronic triggering and alignment, tissue alterations localized in X-ray image data and the coordinates of the selected tissue alterations Z are determined with the aid of image processing algorithms. A manual alignment with the tissue alteration Z is likewise possible. The determined coordinates are transferred to the control unit SEMA in order to align the marker unit MA. The image analysis unit BAE can also be a component of further diagnostic units which are in communication with the mammography system. By use of the image analysis unit BAE and the X-ray images required for the purpose the tissue of the object O fixed here on a working platform AP by means of the compression unit KP, for example the breast O of a patient, is searched for abnormalities and, should any such abnormalities be identified, the coordinates of the tissue alteration Z relating to the analyzed image material are saved and where applicable marked in the 2D or 3D image material. The compression plate KP required for the biopsy has windows or openings in the contact area facing the object O, through which the tissue of the object O is directly accessible by the biopsy needle of the biopsy needle holder BNLH. In this example a working platform AP is additionally illustrated, the height of which is likewise adjustable. The object can be fixed by use of the working platform AP in conjunction with the compression plate KP in such a manner that a biopsy sample can be securely taken. The point-type laser L1 offers the advantage that it gives the alignment of the biopsy needle and optically marks a treatment point BP. An adjustment of the alignment of the first laser L1 is possible for example using mechanical adjustment means in the marker unit MA. As presented in this embodiment, the marker unit MA arranged on the swivel arm SA simultaneously serves as a safety switch offering protection against an accidental insertion of the biopsy needle unit into the biopsy needle holder BNLH since the needle path for positioning the biopsy needle is released only after the marker unit equipped with an optical target device has been removed or folded back.

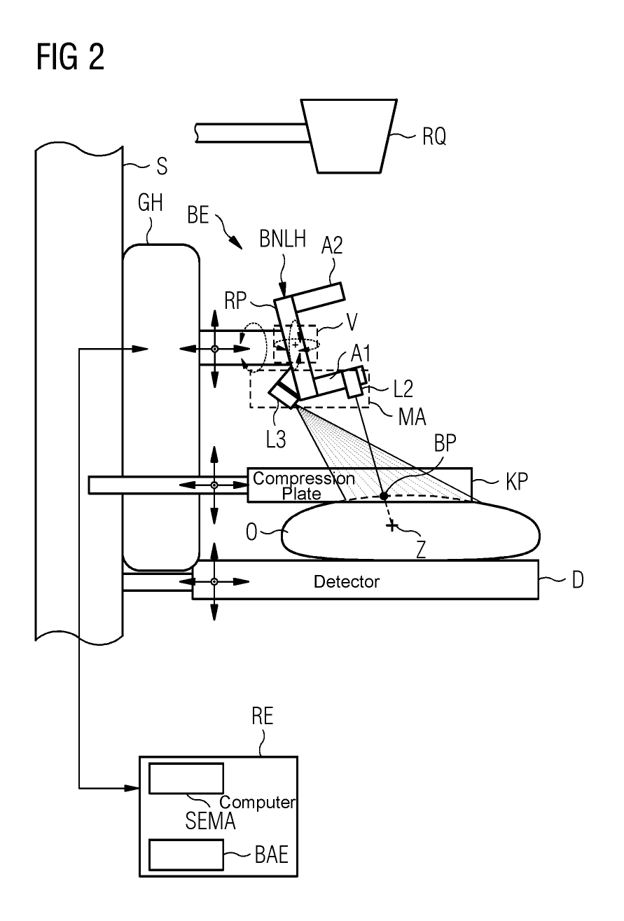

In FIG. 2, a part of a mammography system having the X-ray source RQ which can be fixed on the stand S and can be moved along a trajectory and having the detector unit D which is adjustable at least in height can be seen. The X-ray source RQ and the detector unit D can equally be coupled by way of an arc-shaped retaining element. Furthermore, the biopsy unit BE is also illustrated schematically from the side. The biopsy unit BE is likewise depicted schematically, having a housing GH configured for the positioning of a biopsy needle holder BNLH and accommodating positioning motors for a holding arm H. The units are coordinated or controlled in each case by a computer unit RE associated therewith. For controlling the biopsy unit BE, at least parts of an image analysis unit BAE and also a control unit SEMA for a marker unit MA are provided. The control elements which can be controlled by the control unit SEMA enable a holding arm H on which the biopsy needle holder BNLH is secured and to be positioned in the space. Optionally, a connection unit V configured with a plurality of degrees of freedom can be provided between holding arm H and biopsy needle holder BNLH. The surface of the detector D can serve as a contact area for the breast O of the patient. In FIG. 2 the marker unit MA is configured such that it has a second and a third laser L2, L3. The second and third lasers L2, L3 are configured as a line laser. The marker unit MA is arranged or integrated on or in the first arm A1 pointing to the compression plate KP and also on the rear wall plate RP of the biopsy needle holder BNLH. The second laser L2 is arranged on an arm of the first arm A1 in such a manner that the line laser beam emanating from the second laser L2 displays a first line onto the surface of the object O. The third laser L3 on the reverse side of the rear wall plate RP of the biopsy needle holder BNLH is arranged such that a second line laser beam is emitted from the third laser L3 and the second line laser beam is displayed on the surface of the object O. The first and second line laser beams from the second and third lasers L2, L3 cross each other. The treatment point BP is situated at the crossing point of the two laser lines. An adjustment of the alignment of the second and third lasers L2, L3 can be made for example by using mechanical adjustment means on the respective laser. Optionally, the laser beam described with reference to FIG. 1 and FIG. 2 can be rendered visible with the aid of an atomizer. Disinfectant could be used as an atomizing agent. The alignment of the second and third lasers L2, L3 is effected by the biopsy needle holder either manually and/or under computer control. The marker unit MA is configured such that it remains on the biopsy needle holder BNLH because the second and third lasers L2, L3 are integrated or arranged in or on the lower part of the first arm A1 and also on the lower end of the biopsy needle holder BNLH.

The following is a summary list of reference numerals and the corresponding structure used in the above description of the invention:
S Stand
O Object
AP Working platform
D Detector unit
RQ X-ray source
BE Biopsy unit
GH Housing
H Holding arm
V Connection element
BNLH Biopsy needle holder
RP Rear wall plate
A1 First arm
A2 Second arm
KP Compression plate
SA Swivel arm
BF Fastening element
MA Marker unit
L1 First laser
L2 Second laser
L3 Third laser
LS Laser beam
BP Treatment point
Z Tissue alteration
RE Computer unit
BAE Image analysis unit
SEMA Control module for marker unit

1. A biopsy unit, comprising:
a lockable biopsy needle unit for taking a biopsy;
an alignable biopsy needle holder accommodating said lockable biopsy needle unit; and
a marker unit having at least one marker for specifying a treatment point to be displayed on a surface of an object, wherein coordinates of a determined tissue alteration within the object to be examined are used in order to align said marker unit, and in order to align said marker said alignable biopsy needle holder for said lockable biopsy needle unit is configured such that said least one marker is alignable such that said at least one marker is aligned with the determined tissue alteration within the object and marks the treatment point on the surface of the object.

2. The biopsy unit according to claim 1, wherein said marker unit has at least one optical marker.

3. The biopsy unit according to claim 1, wherein said marker unit emits a light beam predefining a positioning for said lockable biopsy needle unit such that an alignment of a longitudinal axis of a biopsy needle exiting from said lockable biopsy needle unit is predefined by an aligned light beam.

4. The biopsy unit according to claim 1, further comprising a swivel arm for receiving said marker unit.

5. The biopsy unit according to claim 4, wherein said swivel arm is disposed in pivotable fashion on said alignable biopsy needle holder such that said swivel arm prevents an insertion of said lockable biopsy needle unit into said alignable biopsy needle holder while the treatment point is being predefined by said marker unit.

6. The biopsy unit according to claim 2, wherein said optical marker is a point-type laser or is formed from at least two line lasers.

7. The biopsy unit according to claim 1, wherein in order to determine coordinates of tissue alterations stereo image data and/or tomosynthesis image data from a image analysis unit is made available.

8. The biopsy unit according to claim 1, wherein said marker unit has a marker stylus for marking the treatment point.
